(19) United States
(12) Patent Application Publication
(10) Pub. No.: US 2023/0172704 A1
(43) Pub. Date: Jun. 8, 2023

(54) BIOENGINEERED CORNEAL GRAFTS

(71) Applicant: PRECISE BIO 3D LTD, Hevel Modi'in (IL)

(72) Inventors: Ariel EISENBACH, Zufim (IL); Amos EITAN, Jerusalem (IL); Michal MARCUS, Nechalim (IL); Lior ROSENBERG BELMAKER, Shoham (IL); Shai GARTY, Rehovot (IL); Rajesh KUMAR, Temple, TX (US); Lior SHAV, Glen Rock, NJ (US); Aryeh BATT, Har Hebron (IL)

(21) Appl. No.: 18/088,673

(22) Filed: Dec. 26, 2022

Related U.S. Application Data
(63) Continuation of application No. 17/766,670, filed on Apr. 5, 2022.
(60) Provisional application No. 62/911,375, filed on Oct. 7, 2019.
(60) Provisional application No. 62/911,291, filed on Oct. 6, 2019.

(51) Int. Cl. A61F 2/14; B33Y 80/00; A61L 27/24; A61L 27/34; A61L 27/38; A61L 27/52

(52) U.S. Cl. CPC A61F 2/1451; B33Y 80/00; A61L 27/24; A61L 27/34; A61L 27/3813; A61L 27/3834; A61L 27/52; A61F 2240/001

(57) ABSTRACT

The present invention discloses bioengineered corneal grafts for treating either or both Keratoconus and visual impairment, selected from (i) a corneal Onlay comprises or coated by at least one member of Group A, consisting of biocompatible synthetic materials; at least one member of Group B, consisting of at least one type of biological polymer and optionally, at least one member of Group C, consisting of at least one type of protein and (ii) An intrastromal corneal lenticule graft, configured to mimic native corneal stroma tissue by means of its optical properties, mechanical properties, permeability and interaction with corneal stromal cells; wherein at least one portion of said lenticule comprises or coated by at least one member of Group D, consisting of transparent crosslinked hydrogel; at least one member of Group E, consisting of collagen; collagen methacrylate, recombinant mammal collagen, mammal-sourced collagen; and optionally, at least one member of Group F, consisting of Keratocytes and/or stem cells and any combination thereof. The present invention further discloses compositions, methods for production, implementation and treatment of medical indications by aforesaid corneal graft.

### CROSS-REFERENCE

This application is a Continuation Patent Application of U.S. patent application Ser. No. 17/766,670 filed on Apr. 5, 2022, which is a National Phase of international patent application No. PCT/IL2020/051081 filed on Oct. 6, 2020, which claims the benefit of priority of U.S. Provisional Patent Application No. 62/911,291 filed Oct. 6, 2019 and of U.S. Provisional Patent Application No. 62/911,375 filed Oct. 7, 2019, the contents of which are all incorporated herein by reference in their entirety.

### FIELD OF THE INVENTION

The present invention pertains to bioengineered corneal grafts; and more specifically, to corneal Onlays and Inlays. The invention also relates with implantable and biocompatible corneal grafts compositions, to methods for their production and grafting into the cornea and to methods for treating a medical condition of a patient.

### BACKGROUND OF THE INVENTION

Current vision correction techniques include eyeglasses, contact lenses and surgical methodologies which involve cutting and/or removal of tissue, such as laser-assisted in situ keratomileusis (LASIK) and photorefractive keratectomy (PRK). However, surgical methodologies require partially removement of the corneal stroma, which is an irreversible process, might induce complications such as scarring and hazing of the cornea, infections and glary vision. Furthermore, laser surgeries cannot address severe cases of hyperopia, as it requires the removal of a too thick layer from the corneal stroma.

Another approach for optical correction is by adding material to the eye by placing a permanent graft in or on the cornea. A corneal Onlay is a transparent refraction-corrective lens placed near the surface of the eye, directly below the epithelium. Vision correction using Onlay lenses may be preferable to LASIK and/or PRK since it is reversible, repeatable, minimally invasive and easily modifiable. Onlay can serve as an alternative for people whose corneas are too thin for LASIK and/or PRK. Also, in severe cases, Onlay can be combined with LASIK and/or PRK. The Onlay is a thin lenticule (50 to 150 microns thick), which is placed on top of the corneal Bowman's layer, below the epithelium. To some extent, the Onlay is similar to a commercial contact lens, but should not generate an immune response and should promote the growth of a healthy epithelium on top.

Onlay technology, see e.g., U.S. Pat. No. 9,125,735 which is incorporated herein as a reference, is focusing in the vision-correction market, but since it is less invasive than existing laser treatments, it may compete also with contact lenses and eyeglasses solutions.

Several attempts were made to address the Onlay refraction-correction solution. However, they suffer from lack of sufficient permeability or degradation or insufficient epithelium growth on top induce an immune response, and therefore failed in being a satisfying vision-correction solution. Currently, no corneal Onlays are FDA approved in the U.S.

An Onlay, made of a biocompatible material that is highly permeable yet has sufficient surface characteristics to stimulate stable and confirm growth and attachment of the corneal epithelium over its outer surface is still a long-felt need. Also, a stable Onlay which does not to degrade over time, does not induce immune response or scarring of the cornea and administrated via a minimal surgical invasiveness is still required.

Keratoconus is an illness of the cornea, which usually causes a weakening and thinning of the corneal stroma and leads to vision disorders. It affects about 200,000 patients in the US alone. Several treatments exist for Keratoconus, including contact lenses for early stages of the disease, methods of stiffening of the cornea, and corneal transplantation for patients with progressive keratoconus. Other treatments involve the grafting of a synthetic and rigid ring-shaped graft into the corneal stroma.

Hyperopia is the refractive disorder of the human eye, leading to the inability to focus on close objects and therefore results in far-sightless. It affects about 50% of the population in the US above the age of 40, and about 8% of children at age 6. The treatments of hyperopia include regular wearing of eyeglasses or contact lenses, or alternatively, vision-correction laser surgeries as PRK, LASIK, SMILE, and RLE, see e.g., Tran, Khai, and Andrea Ryce. “Laser Refractive Surgery for Vision Correction: A Review of Clinical Effectiveness and Cost-effectiveness.” (2018). However, laser subtractive treatments for hyperopia are limited to low refractive errors patients only, due to the limited thickness of the corneal stroma, see Corbett, Melanie, et al. “Refractive Laser Surgery.” Corneal Topography. Springer, Cham, 2019. 235-268.

Hence, affordable, stable, biocompatible, easily-implantable and patient-tailored corneal grafts are still a long-felt need.

### SUMMARY OF THE INVENTION

The technology of present invention is generally directed to a corneal graft for treating either or both Keratoconus and visual impairment, and selected from (i) an Onlay comprises or coated by at least one member of Group A, consisting of biocompatible synthetic materials; at least one member of Group B, consisting of at least one type of biological polymer; and optionally, at least one member of Group C, consisting of at least one type of protein and (ii) an intrastromal corneal lenticule graft, configured to mimic native corneal stroma tissue by means of its optical properties, mechanical properties, permeability and interaction with corneal stromal cells; wherein at least one portion of the lenticule comprises or coated by at least one member of Group D, consisting of transparent crosslinked hydrogel; at least one member of Group E, consisting of collagen; collagen methacrylate, recombinant mammal collagen, mammal-sourced collagen; and optionally, at least one member of Group F, consisting of Keratocytes and/or stem cells and any combination thereof as disclosed and defined in the present invention.

Some embodiments of the invention disclose the corneal graft as defined above, wherein collagen of Group E is used to make hydrogels of Group D.

Some embodiments of the invention disclose an Onlay. This Onlay comprises or coated by at least one member of Group A, consisting of biocompatible synthetic materials; at least one member of Group B, consisting of at least one type of biological polymer and optionally at least one member of Group C, consisting of at least one type of protein.

Some embodiments of the invention disclose an Onlay as defined above, wherein the Group A consists at least one of the following: 2-(hydroxyethyl)methacrylate (HEMA), HEA, methyl methacrylate (MMA), methacrylic acid (MAA), 2-methacryloyl-oxyethyl phosphorylcholine (MPC), polyethylene glycol (PEG), polycaprolactone (PCL), polyvinyl alcohol (PVA), polyethylene glycol diacrylate, polyethylene diacrylamide, polyethylene glycol dimethacrylate and any mixture or combination thereof

Some embodiments of the invention disclose an Onlay as defined in any of the above, wherein the Group B consists at least one of the following: collagen, recombinant mammals' collagen, collagen methacrylate (ColMA), gelatin, gelatin methacrylate (GelMA), Elastin, mixtures thereof, and combinations thereof.

Some embodiments of the invention disclose an Onlay as defined in any of the above, wherein the Onlay is coated by one or more of the followings: collagen, laminin, fibronectin or a combination thereof, e.g., thereby it is configured to allow epithelium growth.

Some embodiments of the invention disclose an Onlay as defined in any of the above, wherein the Onlay is made in a method consisting of (a) pre-maturating one or more of the followings: seeding stem cells; Limbal stem cells; Epithelial cells on its anterior surface, and then (b) removing the cells prior to grafting.

Some embodiments of the invention disclose an Onlay as defined in any of the above, wherein the Onlay is made in a method consisting of incorporating one or more of the followings: limbal stem cells and epithelial cells, on its anterior surface.

Some embodiments of the invention disclose an Onlay as defined in any of the above, wherein the Onlay is made by one or more techniques selected from a group consisting of molding, 3D-printing, laser-ablating and a combination thereof e.g., thereby providing for optimal and/or patient-specific vision-correction.

Some embodiments of the invention disclose an Onlay as defined in any of the above, wherein the Onlay is made by 3D-printing to shape for optimal and/or patient-specific vision-correction.

Some embodiments of the invention disclose an Onlay as defined in any of the above, wherein the Onlay is made in a method consisting of laser-ablating to the required shape for optimal and/or patient-specific vision-correction.

Some embodiments of the invention disclose an Onlay as defined in any of the above, wherein Onlay comprises sub-micron sized pores. [configured to allow permeability of oxygen, glucose and nutrients].

Some embodiments of the invention disclose an Onlay as defined in any of the above, wherein the Onlay is at least partially made of recombinant mammals, e.g., human collagen.

Some embodiments of the invention disclose an Onlay as defined in any of the above, wherein the Onlay is characterized by that one or more of the following is held true: (a) the Onlay has an optical refractive index which is similar to the native corneal stroma, to avoid light scattering and/or reflections; (b) the Onlay is at least partially blocks UV light; (c) the Onlay is coated by recombinant human collagen; and (d) the Onlay is marked for the correct orientation by a laser engraving, mechanical pressure, pigmented ink, or a combination thereof

It is acknowledged in this respect that refractive index depends on the specific gravity of the hydrogel. The hereto provided refractive index is very close to water (i.e., 1.34) whereas refractive index of the cornea is ranging from 1.34 to 1.38.

Some embodiments of the invention disclose a method for treating visual impairment, comprising a step of grafting an Onlay as defined in any of the above.

Some embodiments of the invention disclose a method for grafting an Onlay as defined in any of the above.

Some embodiments of the invention disclose a method for grafting an Onlay as defined in any of the above, wherein at least one of the following is held true: (a) the method further comprising a step of shaping the Onlay using a laser after grafting; (b) the method further comprising a step of shaping the Onlay using a laser after grafting and a maturation period; e.g., useful in the case of relapsed visual acuity disorder; and (c) the method further comprising a step of utilizing an insertion tool; e.g., to avoid damage of the lenticule and/or the cornea, and to eliminate corneal epithelial cells present inside the cornea after grafting.

Some embodiments of the invention disclose a corneal device for treating visual impairment, comprising an Onlay as defined in any of the above.

Some embodiments of the invention disclose an intrastromal corneal lenticule graft for either or both Keratoconus treatment and vision-correction, configured to mimic native corneal stroma tissue by means of its optical properties, mechanical properties, permeability and interaction with corneal stromal cells; wherein at least one portion of the lenticule comprises or coated by at least one member of Group D, consisting of transparent crosslinked hydrogel; at least one member of Group E, consisting of collagen; collagen methacrylate, recombinant mammal collagen, mammal-sourced collagen; and optionally, at least one member of Group F, consisting of Keratocytes and/or stem cells and any combination thereof.

Some embodiments of the invention disclose an intrastromal corneal lenticule graft as defined above, configured to at least partially mimic the native tissue.

Some embodiments of the invention disclose an intrastromal corneal lenticule graft as defined in any of the above, wherein the lenticule is configured to integrate with surrounding corneal stroma tissue.

Some embodiments of the invention disclose an intrastromal corneal lenticule graft as defined in any of the above, wherein the lenticule is configured to be at least partially remodeled in the eye and replaced by native tissue in a way that the remodeling is not affecting the lenticule geometry and/or optical functionality.

Some embodiments of the invention disclose an intrastromal corneal lenticule graft as defined in any of the above, wherein the lenticule is at least partially made of recombinant mammal, e.g., human collagen.

Some embodiments of the invention disclose an intrastromal corneal lenticule graft as defined in any of the above, wherein the lenticule is coated by recombinant human collagen.

Some embodiments of the invention disclose an intrastromal corneal lenticule graft as defined in any of the above, wherein the lenticule is at least partially made of mammal, e.g., human sourced collagen.

Some embodiments of the invention disclose an intrastromal corneal lenticule graft as defined in any of the above, wherein the lenticule comprises either or both Keratocytes and/or stem cells.

Some embodiments of the invention disclose an intrastromal corneal lenticule graft as defined in any of the above, wherein the lenticule is configured to be tailored made for a specific patient.

Some embodiments of the invention disclose an intrastromal corneal lenticule graft as defined in any of the above, wherein at least one of the following is held true: (a) the lenticule is configured for a spherical refractive correction in the range between about −10 diopters to about 15 diopters; (b) the lenticule is characterized by a non-spherical shape for astigmatism vision correction; (c) the lenticule is characterized by shape for patient-tailored vision correction; (d) the lenticule has an optical refractive index which is similar to the native corneal stroma, to avoid light scattering and/or reflections; (e) the lenticule has elastic modulus between about 50 kPA and about 13 MPa; (f) the lenticule has permeability to glucose, oxygen, and proteins which is comparable to native corneal stroma tissue, i.e., having a permeability which is in the range of about 50% to about 200% of an healthy cornea; (g) the lenticule is configured to make possible the migration of corneal stroma cells such as Keratocytes into the lenticule; and (h) the lenticule at least partially blocks UV light.

Some embodiments of the invention disclose a method for the production of an intrastromal corneal lenticule graft for either or both Keratoconus treatment and vision-correction, configured to mimic native corneal stroma tissue by means of its optical properties, mechanical properties, permeability and interaction with corneal stromal cells; wherein the method comprising steps of (a) providing at least one portion of the lenticule to comprise or to be coated by at least one member of Group D consisting of transparent crosslinked hydrogel; at least one member of Group E, consisting of collagen; collagen solution, collagen methacrylate, recombinant mammal collagen, mammal-sourced collagen; and optionally, at least one member of Group F, consisting of Keratocytes and/or stem cells and any combination thereof; and (b) processing the same by a method selected from 3D printing, laser ablating, molding, and any combinations thereof

Some embodiments of the invention disclose a method for the production of an intrastromal corneal lenticule graft as defined above. The method further characterized by step of 3D printing and/or molding a collagen solution, and crosslinking the same to form a transparent hydrogel.

Some embodiments of the invention disclose a method for the production of an intrastromal corneal lenticule graft as defined in any of the above. The method further characterized by step of concentrating collagen solution to a predefined value in the rage of about 1 to about 15% w/v.

Some embodiments of the invention disclose a method for the production of an intrastromal corneal lenticule graft as defined in any of the above. The method further characterized by step of centrifuging collagen solution.

Some embodiments of the invention disclose a method for the production of an intrastromal corneal lenticule graft as defined in any of the above. The method further characterized by step of 3D printing the collagen solution to a predefined shape.

Some embodiments of the invention disclose a method for the production of an intrastromal corneal lenticule graft as defined in any of the above. The method further characterized by step of molding of the collagen solution.

Some embodiments of the invention disclose a method for the production of an intrastromal corneal lenticule graft as defined in any of the above; wherein the crosslinking is provided by admixing photo-initiator to the collagen solution and applying light on it.

Some embodiments of the invention disclose a method for the production of an intrastromal corneal lenticule graft as defined in any of the above; wherein the crosslinking is provided by admixing N-(3-dimethylaminopropyl)-N′-ethylcarbodiimide (EDC) and/or N-hydroxysuccin-imide (NHS) to the collagen solution.

Some embodiments of the invention disclose a method for the production of an intrastromal corneal lenticule graft as defined in any of the above; wherein the crosslinking is provided in a controlled temperature and humidity.

Some embodiments of the invention disclose a method for the production of an intrastromal corneal lenticule graft as defined in any of the above; wherein the is provided in a controlled gas mixture environment, other than air.

Some embodiments of the invention disclose a method for the production of an intrastromal corneal lenticule graft as defined in any of the above; wherein the molding is provided by designated tool, having the predefined geometry and surface roughness.

Some embodiments of the invention disclose a method for the production of an intrastromal corneal lenticule graft as defined in any of the above; wherein the tool is made of a material selected from a group consisting of composite material, glass, PP, PE, PET, PDMS, PTFE, FEP, and any combination thereof.

Some embodiments of the invention disclose a method for the production of an intrastromal corneal lenticule graft as defined in any of the above; wherein the solution is provided to form hydrogel with an optical refractive index which is similar to the native corneal stroma, thereby configured to avoid light scattering and/or reflections.

Some embodiments of the invention disclose a method for the production of an intrastromal corneal lenticule graft as defined in any of the above; wherein the at least one of the following is held true: (a) the solution is configured to form a hydrogel characterized by elastic modulus ranging between about 50 kPA to about 13 MPa; (b) the solution is configured to form a hydrogel characterized with permeability to glucose, oxygen and proteins which is at least about 50% of the permeability of native corneal stroma tissue; (c) the solution is configured to form a hydrogel which at least partially blocks UV light.

Some embodiments of the invention disclose a method of implementing an intrastromal corneal lenticule graft as defined in any of the above.

Some embodiments of the invention disclose a method of implementing an intrastromal corneal lenticule graft as defined in any of the above; wherein prior to grafting, a step of marking the transparent crosslinked hydrogel for the correct orientation is provided by one or more members of a group consisting of a laser engraver, mechanical press, pigmented ink, or a combination thereof

Some embodiments of the invention disclose methods of grafting the intrastromal lenticule comprising a step of using a laser system, including an excimer laser, e.g., Dippel, Eric J., et al. “Randomized controlled study of excimer laser atherectomy for treatment of femoropopliteal in-stent restenosis: initial results from the EXCITE ISR trial (EXCImer Laser Randomized Controlled Study for Treatment of Femoropopliteal In-Stent Restenosis)” JACC: Cardiovascular Interventions 8.1 Part A (2015): 92-101; and/or femtosecond laser, e.g., Soong H. K. and Malta J. B “Femtosecond lasers in ophthalmology” Am J Ophthalmol 2009;147:189-197.

Some embodiments of the invention disclose a method of implementing an intrastromal corneal lenticule graft as defined in any of the above, comprising step ablating the lenticule by laser to shape and size.

Some embodiments of the invention disclose a method of implementing an intrastromal corneal lenticule graft as defined in any of the above, comprising scanning the lenticule by an OCT, simultaneously to the step of ablating, hence forming a closed-loop feedback mechanism.

Some embodiments of the invention disclose a method of implementing an intrastromal corneal lenticule graft as defined in any of the above; wherein the laser system comprises excimer and/or a femtosecond laser.

Some embodiments of the invention disclose a method of grafting an intrastromal corneal lenticule graft as defined in any of the above, comprising a step of using a laser system, including an excimer laser and/or a femtosecond laser.

Some embodiments of the invention disclose a method of grafting an intrastromal corneal lenticule graft as defined in any of the above, wherein the laser system is used to create a corneal pocket.

Some embodiments of the invention disclose a method of grafting an intrastromal corneal lenticule graft as defined in any of the above, comprising a step of shaping the intrastromal lenticule, by means of laser within patient's cornea, after grafting the same.

Some embodiments of the invention disclose a method of grafting an intrastromal corneal lenticule graft as defined in any of the above, comprising step of optimizing depth and position within the cornea, which was optimized based on OCT scans and mechanical properties measurement of the cornea.

Some embodiments of the invention disclose a method of grafting an intrastromal corneal lenticule graft as defined in any of the above, comprising step of utilizing an insertion tool to avoid damage of the lenticule and/or the cornea, and to eliminate corneal epithelial cells present inside the cornea after grafting.

Some embodiments of the invention disclose a method of grafting an intrastromal corneal lenticule graft as defined in any of the above, wherein the grafting of the lenticule is provided before or after a corneal crosslinking.

### BRIEF DESCRIPTION OF THE DRAWINGS

The various features and advantages of the present invention may be more readily understood with reference to the following detailed description taken in conjunction with the accompanying drawings in which

FIG. 1 schematically illustrates a corneal Onlay according one embodiment of the present invention;

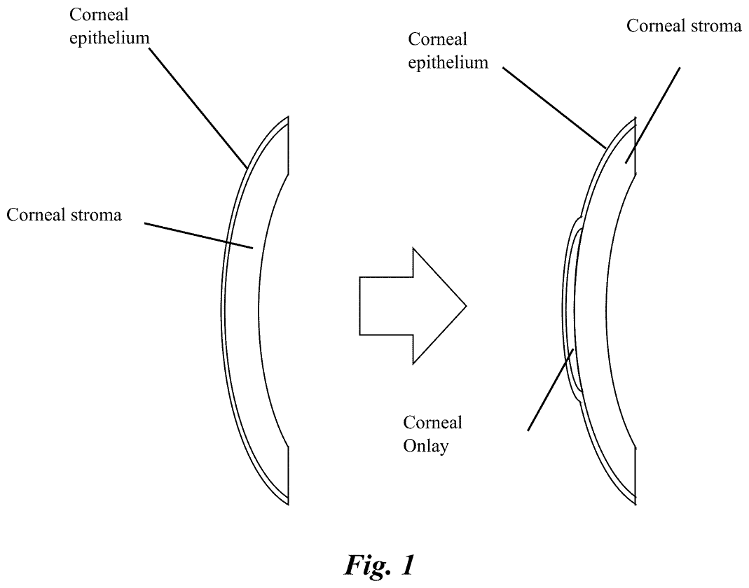

FIG. 2 schematically illustrates a corneal intrastromal lenticule graft according to one embodiment of the present invention, reference is made to currently available link https://commons.wikimedia.org/wiki/File:Hypermetropia.svg which is incorporated herein; and

FIG. 3 shows OCT scans of an intrastromal lenticule graft before and after laser processing, in accordance with a few embodiments of the present invention.

### DETAILED DESCRIPTION OF THE PREFERRED EMBODIMENTS

It is according to a few embodiments of the invention wherein the hereto disclosed technology provides novel corneal grafts; and more specifically, bioengineered corneal Onlays and Inlays. The invention also discloses novel corneal grafts' compositions, methods for their production and inserting, transplanting and/or grafting into the cornea, and methods for treating a medical condition of a patient.

While this invention has been described with respect to various specific examples and embodiments, it is to be understood that the invention is not limited thereto and other embodiments are within the scope of the invention.

The terms “corneal graft” and “corneal implant” interchangeably refer hereinafter to a bioengineered construct that is designed to be implantable in a mammalian (e.g., human) eye and to have properties of at least part of a cornea. The terms further refer to either and both corneal Inlays and Onlays. Cornea transplant (keratoplasty) is a surgical procedure to modify properties of part of the cornea with corneal tissue from a donor or a “synthetic” cornea or portions thereof

The term “Inlay” “intrastromal corneal lenticule graft” interchangeably refer to an intracorneal Inlay, e.g., an alloplastic lenticule placed at the interface of the free corneal cap and the stromal bed or in a corneal pocket.

The term “Onlay” refers to corneal graft, namely an implantable lens or portion thereof, that is placed between Bowman's membrane of the cornea of an eye and the corneal epithelium of the eye.

The term “grafting” and “implanting” interchangeably refers, when relevant, to medical procedures of inserting, grafting, and transplanting of corneal Inlay or corneal Onlay onto or into the cornea.

Unless otherwise stated, with reference to numerical quantities, the term “about” refers to a tolerance of ±25% about the stated nominal value.

A first aspect of the invention, is to disclose affordable, stable, biocompatible, easily-implantable and patient-tailored corneal Onlay:

It is according to a few embodiments of the invention wherein the Onlay comprises or coated by at least one member of Group A, consisting of biocompatible synthetic materials; at least one member of Group B, consisting of at least one type of biological polymer and optionally at least one member of Group C, consisting of at least one type of protein.

It is according to a few embodiments of the invention wherein the thicknesses of the grafted member is ranging between about 30 to about 200 microns. Diameter of about 7 mm. Refractive correction is ranging from about −15 D to about +20 D, spherical or with astigmatism correction. Possibly having thinner periphery. Possibly incorporating marks for astigmatism axis alignment.

It is according to yet other embodiments of the invention wherein the Onlay is made of natural occurring compositions, including polymers. Such polymers are selected from a group consisting, inter alia, collagen and/or collagen methacrylate, human, animal or recombinant source, e.g., about 1 to about 15% w/v; fibronectin; elastin, laminin and any combination thereof.

It is according to other embodiments of the invention wherein biocompatible synthetic polymers are provided useful, including polymers that are selected from a group consisting, inter alia, 2-hydroxyl methacrylate (HEMA); 2-hydroxyethylacrylate (HEA); Methyl methacrylate (MMA); Methacrylic acid (MAA); Methacryloyloxyethyl phosphorylcholine (MPC); Poly(ethylene glycol) (PEG) and/or Poly(ethylene glycol) diacrylate (PEGDA) and/or poly(ethylene glycol) dimethacrylate (PEGDMA), Multi-arm Poly(ethylene glycol) diacrylate; Poly(ε-caprolactone) (PCL); Poly(vinyl alcohol) (PVA); Photo-initiator LAP; Irgacure 2959; APS-TEMED and any combination thereof.

It is according to other embodiments of the invention wherein the Onlay is characterized by the following parameters: it is transparent (e.g., more than 85% visible light transmission, less than 5% haze); stiffness similar to natural cornea (about 50 kPa to about 13 MPa), to enable healthy epithelial cells growth; it is permeable to glucose and proteins, similarly to native corneal stroma and not less than about 10\hat{ }−6 cm\hat{ }2 per sec for glucose; it enables epithelium cells growth on anterior surface and the formation of healthy epithelial layers; it comprises high water content, it is non-degradable or has long term stability; it is biocompatible with low immune response, not induce fibrosis, and it can be shaped with laser processing and/or 3D printing or molding.

It is according to other embodiments of the invention wherein the Onlay is substantially mainly synthetic, as the enzymatic activity in the epithelium is relatively high, and natural hydrogel would be degraded significantly after grafting. It is possible that Onlay based on natural compounds would be remodeled by the body and form native tissue.

In order to improve vision in presbyopia, myopia, hyperopia and astigmatism patients, a transparent, permeable hydrogel can be grafted into the cornea, below the epithelium layer, to fix the curvature of the cornea. Onlays can be referred to as permanent bio-compatible contact lenses. The development of an Onlay graft has two main challenges: (1) the surface of the Onlay should be protein-based in order to enable normal epithelium growth above it. Yet, the Onlay needs to be non-degradable in order achieve stable vision correction; and, (2) the Onlay must be permeable to glucose and nutrients in order to nourish the epithelium layer. A healthy epithelium is essential as it acts as a protective barrier to keep bacteria, dust and other foreign substances from penetrating the eye.

The combination between natural and synthetic polymers disclosed herein are designed to address these challenges and form a viable solution for refraction-corrective Onlays. In preferred embodiments, the Onlay comprises a hydrogel layer and a coating layer. The hydrogel layer is made of biocompatible synthetic polymer, with additional bio-mimicking substances, and the coating is made of biological proteins which present binding sites and other nutrients and indicators for the epithelial cells.

The Onlay lens graft can be molded, 3D printed, or laser ablated to the required curvature and geometry. In some cases, the grafted lenticules are designed specifically to the patient, allowing better correction of the refractive disorder.

It is according to other embodiments of the invention wherein the preparation methods consists, inter alia, the following steps: (a) mixing materials; (b) injecting into mold and/or 3D printing; (c) photo and/or thermal polymerization; (d) washing out residues; (e) possibly laser processing; (f) possibly applying an additional coating layer to allow cells growth; (g) possibly seeding epithelial and/or limbal and/or stem cells on top of the Onlay for a maturation period (see below); (h) testing (refraction, transparency, homogeneity etc.); and (i) applying thrombin and/or fibrinogen on the posterior side

The maturation-period is provided herein useful for seed cells in vitro on top of the Onlay, after or instead the coating process, and let them mature the Onlay and produce binding sites and/or ECM and/or collagen for better and faster epithelization after grafting. After the maturation period the cells are removed to minimize any immune response, and the Onlay is tested and grafted.

It is according to other embodiments of the invention wherein graft of the Onlay is provided useful by the following method: (a) removing the epithelium and exposing the Bowman's layer; (b) applying fibrinogen and/or thrombin on the Bowman's layer; (c) aligning and attaching the Onlay to the cornea for about 10 to about 60 seconds, possibly with a designated tool; (d) Possibly applying protective contact lens to prevent dry eyes and infections; and (e) possibly post-treating with nutrients, steroids, antibiotics and lubricants for up to four weeks.

It is according to other embodiments of the invention wherein shaping is provided by one of the following techniques: Laser processing of the lenticule before grafting, according to patient's OCT scan; Laser processing of the lenticule immediately after grafting; and/or Laser processing of the lenticule after a maturation period (about 1 day to about 12 weeks), during which the cornea heals and possibly changes its geometry.

As an example, and in a non-limiting manner, an Onlay according to an embodiment of is at least partially made of a combination of biocompatible synthetic materials and biological polymers and proteins as defined below, such that at least one of the following is being held true: (a) the Onlay is made by composition selected from a group consisting HEMA, HEA, MAA, MMA, MPC, PEG, PCL, PVA or any mixture or combination thereof, and collagen, ColMA, gelatin, GelMA, Elastin or any mixture or combination thereof; (b) the Onlay is coated with collagen, laminin or fibronectin to allow epithelium growth; (c) the Onlay is made in a method consisting of a pre-maturation step of seeding stem cells; Limbal stem cells; Epithelial cells on its anterior surface, and removing the cells prior to grafting; (e) the Onlay is made in a method consisting of incorporating limbal stem cells and/or epithelial cells on its anterior surface; (f) the Onlay is made in a method consisting of is 3D-printing to the required shape for optimal and/or patient-specific vision-correction; (g) the Onlay is made in a method consisting of laser-ablating to the required shape for optimal and/or patient-specific vision-correction; (h) after grafting, the Onlay is processed using laser systems to the required shape for optimal and/or patient-specific vision-correction; (i) the Onlay comprises sub-micron sized pores to allow permeability of glucose and nutrients. Other embodiments disclose a method for improving a person's vision, comprising a step of grafting an Onlay as defined in any of above and a corneal device for improving a person's vision, comprising an Onlay as defined in any of the above.

As an example, and in a non-limiting manner, an Onlay is made of a composition ranging from 10 to 30% HEMA and 5 to 25% HEA, and 0.2 to 2% collagen methacrylate (w/v), with 0.1 to 1% Irgacure 2959 as a photo-crosslinker.

In order to produce Onlays, the following methods were used: (a) materials are thoroughly mixed in aqueous solution and centrifuged for bubbles extraction; (b) The composition is then injected into a transparent glass mold, which designed to form a 200 to 300 microns thick hydrogel, with no optical power, but having a base curve radius of 8.0 to 10.0 to fit corneal anterior surface curvature. (c) Mold is then inserted into a crosslinking light chamber for 5 to 10 minutes at irradiance of 5 to 10 mW per cm\hat{ }2. (d) Yielded crosslinked hydrogel is then washed for 24 to 72 h in aqueous solution to remove residues. (e) The hereto washed hydrogel is then placed within an excimer laser system, and ablated to form a lenticule with an optical power according to an OCT scan of the patient's eyes. (f) The resulting Onlay lenticule is scanned by an OCT system to assure its optical properties. (g) lenticules are possibly marked using Gentian Violet surgical marker, by an arrow shaped and an “S” shaped marks, for easier grafting orientation. Prior to implantation, Onlay are submerged in 0.1 mg per ml collagen solution for 30 to 60 minutes, followed by several gentle washes.

A second aspect of the invention, is to disclose affordable, stable, biocompatible, easily-implantable and patient-tailored corneal Inlay, also termed as intrastromal corneal lenticule graft:

It is according to other embodiments of the invention wherein intrastromal corneal lenticule graft is provided useful for either or both Keratoconus treatment and vision-correction. The intrastromal corneal lenticule graft is configured to mimic native corneal stroma tissue by means of its optical properties, mechanical properties, permeability and interaction with corneal stromal cells. In this intrastromal corneal lenticule graft, at least one portion of the lenticule comprises or coated by at least one member of Group D, consisting of transparent crosslinked hydrogel; at least one member of Group E, consisting of collagen; collagen methacrylate, recombinant mammal collagen, mammal-sourced collagen; and optionally, at least one member of Group F, consisting of Keratocytes and/or stem cells and any combination thereof.

In order to improve vision in keratoconus and hyperopia patients, a collagen-based hydrogel of the present invention can be grafted into the corneal stroma, mimicking the functionality and characteristics of the natural tissue. In some cases, the grafted lenticules are designed specifically for the patient, allowing better correction of the refractive disorder. In some embodiments of the current invention, the graft is made of recombinant human collagen with additional proteins and polymers, allowing the graft to integrate and fuse with surrounding tissue over time. In some embodiments of the current invention, stem cells or keratocytes cells are incorporated into or on the surface of the graft. In some cases, the graft can be grafted into a corneal flap made by a laser. In some embodiments of the current invention, the graft is molded, 3D printed, or laser ablated to the required curvature and geometry.

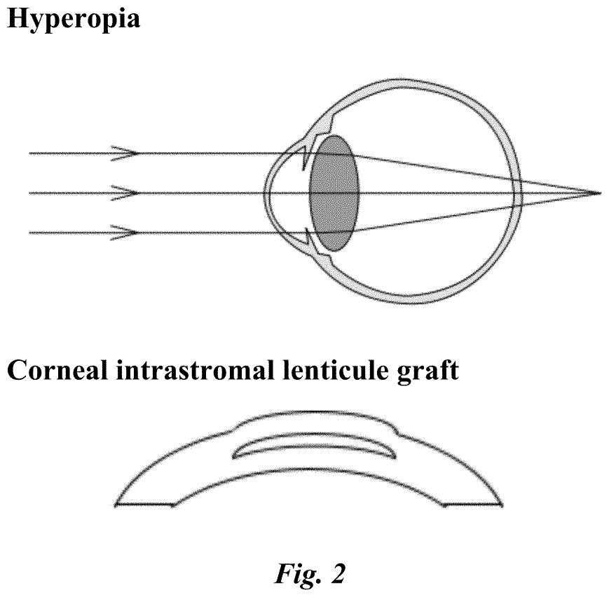

Reference is now made to FIG. 2 which schematically illustrates an intrastromal corneal lenticule graft according to a few embodiments of the present invention.

It is according to a few embodiments of the invention wherein the intrastromal lenticule graft is characterized by thicknesses ranging between about 30 to about 250 microns. Diameter is ranging from about 4 to about 9 mm. Refractive correction varies from about −10 D to about +15 D, spherical or with astigmatism correction, or customized for a specific patient's corneal condition. The lenticule is characterized by a possible thinner periphery and/or a possible mark for astigmatism axis alignment.

In a few embodiments of the invention, intrastromal lenticule is made by compositions comprising collagen. The collagen is selected from a group consisting of collagen and/or Collagen methacrylate; collagen of human, animal, or recombinant source, e.g., from about 1% to about 15% w/v., preferably between about 6 and about 13% w/v, where native corneal stroma has about 13% collagen.

In a few embodiments of the invention, intrastromal lenticule is made by compositions comprising additional natural polymers. Including those selected from a group consisting of gelatin and/or gelatin methacrylate, hyaluronic acid (HA) and/or N-(2-hydroxypropyl) methacrylamide (HAMA), elastin, fibronectin, or a mixture thereof

In a few embodiments of the invention, intrastromal lenticule is made by compositions comprising biocompatible synthetic polymers. including those selected from a group consisting of PEG and its derivatives (PEGDA, PEGDMA, multi-arm PEG); 2-Hydroxyethyl methacrylate HEMA; HEA; PCL; poly(lactic-co-glycolic acid) (PLGA); MPC; and/or additional proteins, such as Laminin, as defined in Aumailley, Monique, et al. “A simplified laminin nomenclature,” Matrix biology 24.5 (2005): 326-332.

In a few embodiments of the invention, intrastromal lenticule is made by utilizing photo-initiator(s) crosslinker, such as LAP, and commercially available Irgacure 2959 product by Sigma-Aldrich, USA. It possibly may comprise biocompatible dye for easy handling. In other embodiments of the invention, the lenticule is made utilizing other crosslinkers, such as EDC and/or NHS molecules.

It is according to a few embodiments of the invention wherein the invention as disclosed here provided useful means, compositions and methods of corneal transplantation or corneal Inlay grafting with cross-linking for preventing an immune response to a corneal graft and/or rejection of the corneal graft by the patient, and for preventing vascular and/or fibrous tissue growth on, and surrounding a keratoprosthesis lens or other type of corneal graft or Inlay.

It is according to a few embodiments of the invention wherein the intrastromal lenticule graft of the present invention is characterized by the following parameters: it is transparent (<85% visible light transmission, >3% haze); its stiffness is similar to central corneal stroma (from about 50 kPa to about 13 MPa, e.g., about 15 0kPa); it is stiff enough to allow the grafting procedure; it is permeable to glucose, oxygen, and proteins; it has high water content; it is non-degradable or slow degradable and can be slowly remodeled by the body and replaced with native tissue without affecting its geometry and optical properties; it is not stimulating an immune response and can be shaped with laser processing and/or 3D printing or molding.

It is according to a few embodiments of the invention wherein the intrastromal lenticule of the present invention is prepared by various methods, including those selected from a group consisting of the following steps: mixing materials; injecting into mold and/or pressing in mold and/or 3D printing; UV and/or thermal and/or chemical casting; washing out residues; possibly additional step(s) of laser processing; coating with soaking in collagen and/or proteins and/or nutrients

It is according to a few embodiments of the invention wherein the intrastromal lenticule of the present invention is grafted by various other methods, including those selected from a group consisting steps of providing a corneal flap using a mechanical tool or PRK and/or LASIK excimer laser; providing a corneal pocket using a mechanical tool or femtosecond laser (e.g., SMILE procedure); possibly to use a designated tool and/or viscoelastic material for the insertion and alignment in the pocket.

As an example, and in a non-limiting manner, an Inlay is made by lyophilized human collagen type I, mixed in 20 mM hydrochloric acid to get 6% (w/v) collagen solution. Additional 1 to 10% v/v HEA solution is mixed with the collagen, and 1-ethyl-3-(3-dimethylaminopropyl)-carbodiimide (EDC) is admixed to form 0.5% (w/v) solution. The yield is thoroughly mixed and centrifuged, and poured into poly propylene (PP) contact lens molds. The molds are inserted into nitrogen chambers at 10 degrees C. for 12 to 24 hours. After crosslinking, lenticules are washed in saline for 24 to 72 hours. Washed hydrogel is then placed in an Excimer Laser system and ablated to form a lenticule with optical power according to an OCT scan of the patient's eyes. The resulting Inlay lenticule is further scanned by an OCT system to assure its optical properties, and possibly, lenticules are marked using Gentian Violet surgical marker by an arrow shaped and an “S” shaped marks, for easier grafting orientation.

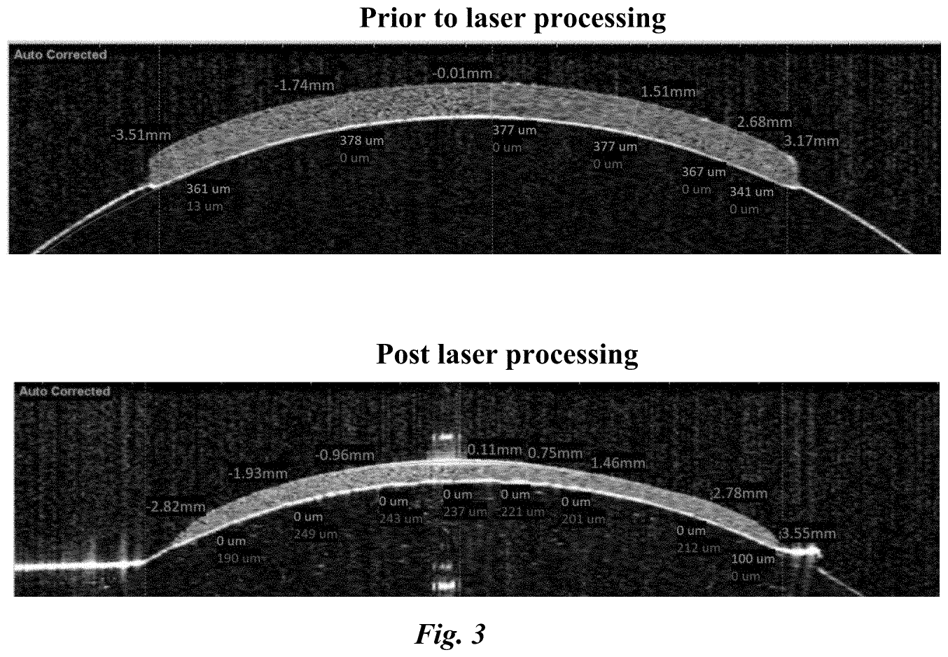

Reference is now made to FIG. 3 which shows OCT scans of an intrastromal lenticule graft before and after laser processing, in accordance with a few embodiments of the present invention.

Several publications, patents, and patent applications have been cited hereinabove. Each of the cited publications, patents, and patent applications are hereby incorporated by reference in their entireties.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A corneal lenticule graft for treating either or both Keratoconus and visual impairment, corneal, configured to mimic native corneal stroma tissue by means of its optical properties, mechanical properties, permeability and interaction with corneal stromal cells; wherein at least one portion of said lenticule comprises or coated at least one member selected from a group consisting of collagen; collagen methacrylate, recombinant mammal collagen, mammal-sourced collagen; and any combination thereof.

2. The corneal lenticule graft of claim 1, additionally comprising at least one biocompatible synthetic material selected from a group consisting of HEMA, HEA, MAA, MMA, MPC, PEG, PCL, PVA and any mixture or combination thereof.

3. The corneal lenticule graft of claim 1, additionally comprising at least one biocompatible synthetic materials selected from a group consisting of ColMA, gelatin, GelMA, Elastin and any mixture or combination thereof.

4. The corneal lenticule graft of claim 1, coated by one or more of the followings:
collagen, laminin fibronectin or a combination thereof.

5. The corneal lenticule graft of claim 1, wherein said corneal lenticule graft is made by one or more techniques selected from a group consisting of molding, 3D-printing, laser-ablating and a combination thereof

6. The corneal lenticule graft of claim 1, wherein said corneal lenticule graft comprises sub-micron sized pores.

7. The corneal lenticule graft of claim 1, wherein said corneal lenticule graft is coated by recombinant human collagen.

8. The corneal lenticule graft of claim 1, characterized by that one or more of the following is held true: a. said corneal lenticule graft has an optical refractive index which is similar to the native corneal stroma, to avoid light scattering and/or reflections; b. said corneal lenticule graft is at least partially blocks UV light; and c. said corneal lenticule graft is marked for the correct orientation by a laser engraving, mechanical pressure, pigmented ink, or a combination thereof.

9. The corneal lenticule graft of claim 1, wherein said corneal lenticule graft is crosslinked.

10. The corneal lenticule graft of claim 1, wherein said corneal lenticule graft is crosslinked by admixing photoinitiator to said corneal lenticule graft and applying light on it.

11. The corneal lenticule graft of claim 9, wherein said crosslinking is provided by admixing EDC and/or NHS molecules to said corneal lenticule graft.

12. The corneal lenticule graft of claim 9, wherein said crosslinking is provided in a controlled temperature and humidity.

13. The corneal lenticule graft of claim 9, wherein said corneal lenticule graft is lyophilized prior to the same being crosslinked.

14. A method for treating visual impairment, comprising a step of grafting at least one corneal lenticule graft of claim 1.

15. The method of claim 14, wherein at least one of the following is held true: a. said method further comprising a step of shaping said corneal lenticule graft using a laser after grafting; b. said method further comprising a step of shaping said corneal lenticule graft using a laser after grafting and a maturation period; and c. said method further comprising a step of utilizing an insertion tool.

16. The corneal lenticule graft of claim 1, wherein at least one of the following is held true: a. said lenticule is configured for a spherical refractive correction in the range between about −10 diopters to about 15 diopters; b. said lenticule is characterized by a non-spherical shape for astigmatism vision correction; c. said lenticule is characterized by shape for patient-tailored vision correction; d. said lenticule has an optical refractive index which is similar to the native corneal stroma, to avoid light scattering and/or reflections; e. said lenticule has elastic modulus between about 50 kPA and about 13 MPa; f. said lenticule has permeability to glucose, oxygen, and proteins which is comparable to native corneal stroma tissue; g. said lenticule is configured to make possible the migration of corneal stroma cells such as Keratocytes into said lenticule; and h. said lenticule at least partially blocks UV light.

17. A method for the production of an intrastromal corneal lenticule graft for either or both Keratoconus treatment and vision-correction, configured to mimic native corneal stroma tissue by means of its optical properties, mechanical properties, permeability and interaction with corneal stromal cells; wherein said method comprising steps of a. providing at least one portion of said lenticule to comprise or to be coated by at least one selected from a group consisting of collagen; collagen solution, collagen methacrylate, recombinant mammal collagen, mammal-sourced collagen and any combination thereof; and, b. processing the same by a method selected from 3D printing, laser ablating, molding, and any combinations thereof

18. The method of claim 17, additionally comprising step of providing at least one portion of said lenticule to comprise or to be coated by at least one selected from a group consisting Keratocytes and/or stem cells and any combination thereof

19. The method of claim 17, characterized by step of 3D printing and/or molding a collagen solution, and crosslinking the same to form a transparent hydrogel.

20. The method of claim 17, characterized by step of concentrating collagen solution to a predefined value in the rage of about 1 to about 15% w/v.

21. The method of claim 17, characterized by step of centrifuging collagen solution.

22. The method of claim 17, characterized by step of 3D printing the collagen solution to a predefined shape.

23. The method of claim 17, characterized by step of molding of said collagen solution.

24. The method of claim 19, wherein said crosslinking is provided by admixing photoinitiator to said collagen solution and applying light on it.

25. The method of claim 19, wherein said crosslinking is provided by admixing EDC and/or NHS molecules to said collagen solution.

26. The method of claim 19, wherein said crosslinking is provided in a controlled temperature and humidity.

27. The method of claim 18, additionally comprising step of lyophilizing said intrastromal corneal lenticule graft prior to said step of crosslinking said corneal lenticule graft.

28. The method of claim 19, wherein said crosslinking is provided in a controlled gas mixture environment, other than air.

29. The method of claim 17, wherein said molding is provided by designated tool, having the predefined geometry and surface roughness.

30. The method of claim 29, wherein said tool is made of a material selected from a group consisting of composite material, glass, PP, PE, PET, PDMS, PTFE, FEP, and any combination thereof

31. The method of claim 17, wherein said solution is provided to form hydrogel with an optical refractive index which is similar to the native corneal stroma, thereby configured to avoid light scattering and/or reflections.

32. The method of claim 17, wherein at least one of the following is held true: a. said solution is configured to form a hydrogel characterized by elastic modulus ranging between about 50 kPA to about 13 MPa; b. said solution is configured to form a hydrogel characterized with permeability to glucose, oxygen and proteins which is at least about 50% of the permeability of native corneal stroma tissue; c. said solution is configured to form a hydrogel which at least partially blocks UV light.

33. The method of claim 17, additionally comprising step of marking said transparent crosslinked hydrogel for the correct orientation by one or more members of a group consisting of a laser engraver, mechanical press, pigmented ink, or a combination thereof.

34. The method of claim 17, comprising step ablating said lenticule by laser to shape and size.

35. The method of claim 34, comprising scanning said lenticule by an OCT, simultaneously to said step of ablating, hence forming a closed-loop feedback mechanism.

36. The method of claim 34, wherein said laser system comprises excimer and/or a femtosecond laser.

37. A method of grafting an intrastromal corneal lenticule graft as defined in claim 1, comprising a step of using a laser system, including an Excimer Laser and/or a femtosecond laser.

38. The method of claim 37, comprising a step of shaping said intrastromal lenticule, by means of laser within patient's cornea, after grafting the same.

39. The method of claim 37, comprising step of optimizing depth and position within the cornea, which was optimized based on OCT scans and mechanical properties measurement of the cornea.

40. The method of claim 37, wherein said of grafting said lenticule is provided before or after a corneal crosslinking.
